(19) United States
(12) Patent Application Publication
(10) Pub. No.: US 2015/0126773 A1
(43) Pub. Date: May 7, 2015

(54) USE OF SOLUTIONS OF SYNTHETIC ALKYLATED GRAPHENES SOLUTIONS FOR MULTIPHASE FLOW TEST FLUIDS TO BE USED BY THE OIL AND GAS INDUSTRY

(71) Applicant: Sinvent AS, Trondheim (NO)

(72) Inventor: Vincent Olivier Pauchard, Trondheim (NO)

(21) Appl. No.: 14/396,904

(22) PCT Filed: Apr. 25, 2013
(86) PCT No.: PCT/NO2013/050072

§ 371 Date: Oct. 24, 2014

Related U.S. Application Data
(60) Provisional application No. 61/638,046, filed on Apr. 25, 2012.

(51) Int. Cl. C07C 57/40; G01N 33/28; C07C 15/20

(52) U.S. Cl. CPC C07C 57/40; C07C 15/20; G01N 33/28; C07C 2103/54
USPC 562/488; 562/405; 585/26; 585/1

(57) ABSTRACT

Synthetic alkylated graphenes of well chosen structure are prepared and used for mimicking indigenous asphaltenes. The mixtures can be used as multiphase flow test fluids in general and specifically in the Oil and gas industry.

### TECHNICAL FIELD

The application relates to chemicals to be used in multiphase flow experiments in the oil and gas industry.

### BACKGROUND ART

The oil and gas industry is in need of fluids to test/qualify equipments (such as flow meters, pumps, electrocoalescers, hydrocyclones or any other separation device) or models (pipe flow simulations, water separation prediction). Crude oils are often considered too complex for that purpose (very complex chemistry that differs a lot from one field to the other, safety issues, cost of samples etc). On the other hand it is difficult to mimic all relevant properties of crude oils with synthetic model fluids. The main difficulty is to reproduce the indigenous surfactants adsorption behaviour (and its consequences such as emulsion stabilization) with model fluids.

The dispersion properties of crude oil/water mixtures (and in particular emulsion stability) largely originate from the adsorption at the water surface of indigenous surfactants present in the crude oil. For a large share of crude oils, those surfactants are identified as asphaltenes (large polycyclic molecules precipitating upon addition of an alkane to crude oil). The molecular structure and weight of those asphaltenes have been debated during the past decades. A coherent representation has recently emerged: (H. Groenzin, O. C. Mullins, Energy & Fuels 14(2000) 677-684).
Average molecular weight 750 g/mol.
Number of pericondensed aromatic rings around 7-8.
Some alkyl chains grafted on the aromatic core.
Some heteroatoms (S, O, N) in polar groups giving rise to interfacial activity.

However published attempts to synthesize large polycyclic molecules bearing enough polar groups to be surface active has lead to a interfacial conformation of molecules largely differing that of natural asphaltenes. (Andrews, A. B.; McClelland, A.; Korkeila, A.; Demidov, A.; Krummel, A.; Mullins, O. C.; Chen Z. Molecular Orientation of Asphaltenes and PAH Model Compounds in LangmuirBlodgett Films Using Sum Frequency Generation Spectroscopy. Langmuir, 2011, 27 (10), pp 6049-6058)

### SUMMARY OF INVENTION

The invention relates to a synthetic compound characterized in that it comprises a pericondensed aromatic core with 4-12 aromatic rings substituted with 1-2 linear alkyl chains containing 5-12 carbon atoms, having a molecular mass between 300-800 g/mol and wherein the proportion of aliphatic carbon atoms ranges from 30-60% of the total number of carbon atoms in the molecule.

Preferably the compound above has range of molecular mass between 600-800 g/mol, preferably about 750 g/mol.

Preferably the compound above, comprises alkyl chains contain 5-12 carbon atoms, preferably 7-8 carbon atoms.

Preferably the compound above is selected from the group consisting of:

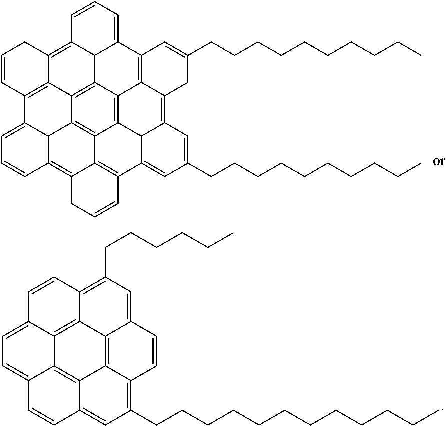

The compounds above could be further substituted by carboxylic functions located on the terminal carbon of the alkyl chain. An atom carbon on the poly-aromatic central core could also be substituted by a nitrogen or sulfur atom. However substitution is not necessary.

The invention also relates to mixtures of the compounds above dissolved in aromatic or aliphatic base oils or mixtures thereof.

Preferably the mixture above according comprise alkylated graphenes 10 to 200 ppm (w/w).

The invention also relates mixtures above as multiphase flow test fluids.

The test fluids above are preferably used for separation tests.

The separation tests above are preferably used in the oil and gas industry.

It is hereafter claimed that the above family of synthetic molecules can be used to mimic asphaltenic oils.

It is hereafter claimed that no polar groups are necessary to give rise to adsorption of asphaltenes and that large polycyclic molecules without heteroatoms can adsorb at the water surface due to the electron donor/acceptor behavior of the aromatic core. It has been verified that both the formation of a rigid skin around water droplets and emulsion stability happen without polar group.

### Technical Problem

As discussed under prior art crude oil and asphaltenes are not well defined and difficult to mimic in multiphase flow testing.

To mimic crude oil, adsorption mechanisms are not sufficient. It is also necessary to mimic the solution properties of asphaltenes in crude oil so that equilibrium (partition) between bulk solution and adsorbed layer. With that respect it has been shown in the literature that per-alkylated hexa-peri-hexabenzocoronene molecules (˜1000 g/mol, see FIG. 1) show significant differences with indigenous asphaltenes in terms of aggregation thresholds, aggregates morphology and precipitation (Energy & Fuels 2006, 20, 2439-2447).

### Solution to Problem

The solution to the problem is the invention as outlined in the claims.

Synthetic alkylated graphenes of well chosen structure (that can be tailored by careful choice of synthesis route) are proposed for mimicking indigenous asphaltenes. Those molecules are dissolved in base oils for producing model fluids.

The mixtures can be used as multiphase flow test fluids in general and specifically in the Oil and gas industry.

The alkylated graphenes are dissolved in commercial base oils of different types (aromatic and aliphatic) in order to adjust:
The solubility of the graphenes.
The density and the viscosity of the solution.

The alkylated graphenes (with possible polar substitutes) concentration is ranging from 10 to 200 ppm only depending upon the type of crude oil to be mimicked. This concentration can be adjusted based upon dynamic interfacial tension and emulsion stability tests.

The synthetic alkylated coronene from FIG. 2 has a behavior similar to natural asphaltenes as indicated in FIGS. 4 to 6.

### Advantageous Effects of Invention

Use of the model compounds as outlined in the claims gives the operator more readily available tools to assess multiphase flow and to compare such flows during production and between different wells.

### BRIEF DESCRIPTION OF DRAWINGS

FIG. 1 shows peralkylated hexa-peri-hexabenzocoronene used for comparison with asphaltenes in Energy & Fuels 2006, 20, 2439-2447 (R are alkyl chains either nonyl of hexyl).

FIG. 2 is an Example of model asphaltenes (˜800 g/mol)

FIG. 3 is an Example of model asphaltenes (˜500 g/mol)

FIG. 4 shows the Dynamic Interfacial Tension of a synthetic asphaltene in toluene solution against deionised water.

FIG. 5 is demonstrating the Morphology of 30% water cut emulsions after 1 day of aging.
(a) 100 ppm synthetic asphaltenes in 30% aromatic/70% aliphatic base oil.
(b) 100 ppm natural asphaltenes in 30% aromatic/70% aliphatic base oil.

FIG. 6 demonstrates the rigid skin appearing upon contraction of a water droplet aged in a synthetic asphaltene solution.

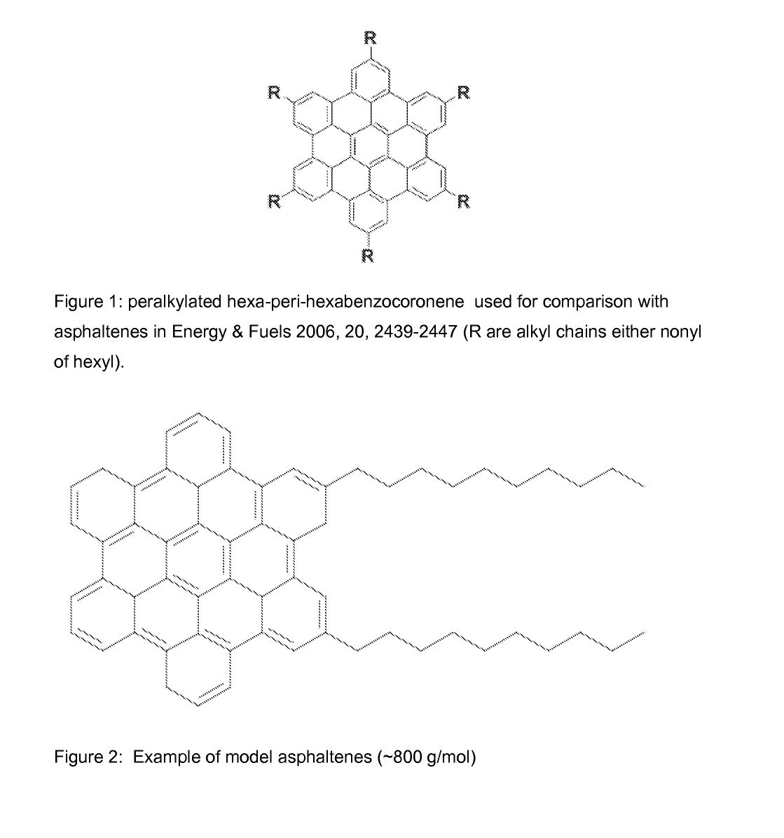

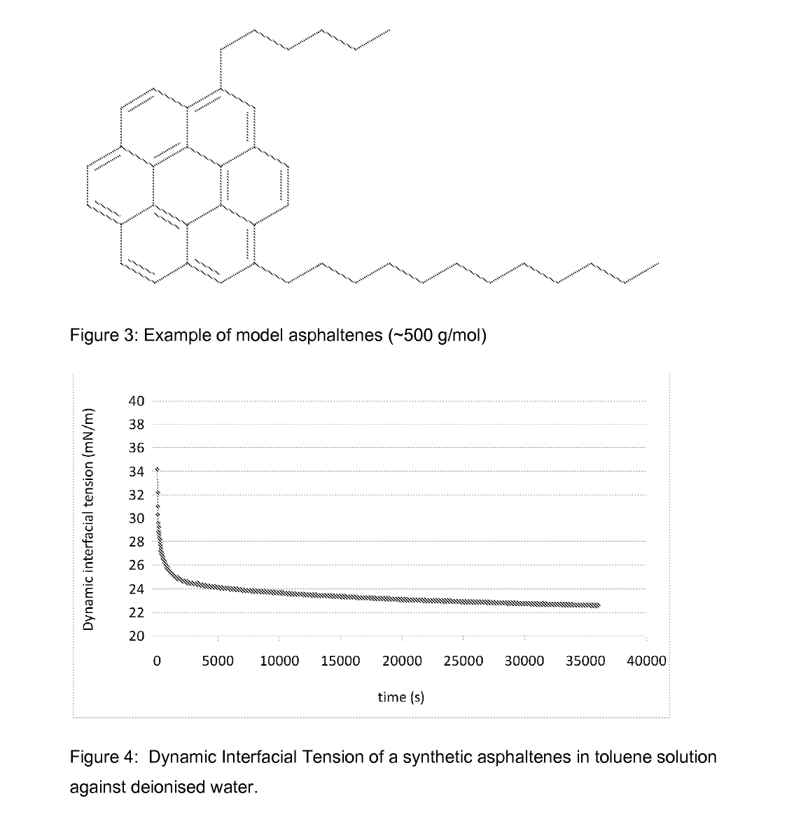

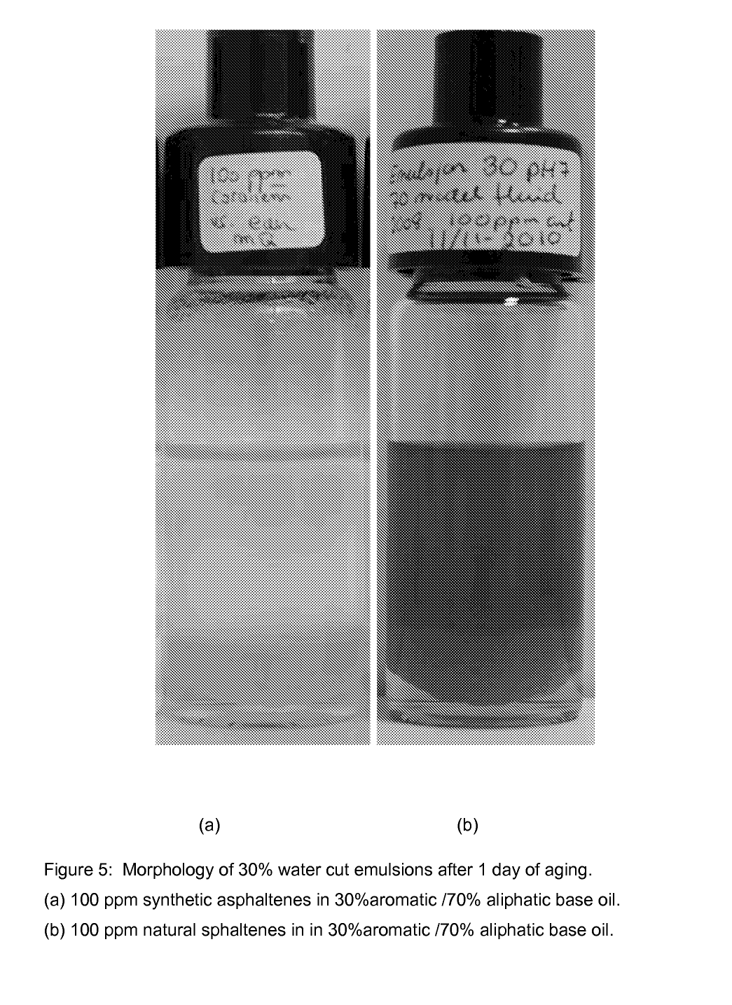

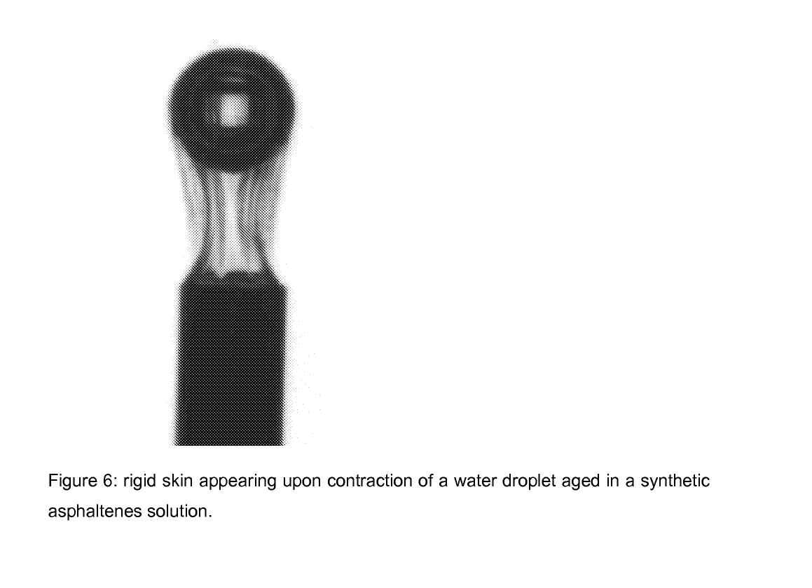

### DESCRIPTION OF EMBODIMENTS

### Examples

### Example 1

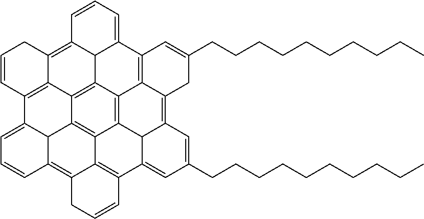 [SMILES: CCCCCCCCCCC1=CC2=C3CC(CCCCCCCCCC)=CC4=C3c3c5c6c7c8c3C2C(=C1)C1=C8C(=C2C=CC=C(C3=C6C(=C6C=CC=C4C65)CC=C3)C27)CC=C1]

Model asphaltene (˜800 g/mol) (see also FIG. 2)

Synthesis Route

a) 1,2-bis(4-bromophenyl)-3,4,5,6-tetraphenylbenzene

A mixture of tetraphenylcyclopentadienone (5.0 mmol), and bis(p-promophenyl) acetylene (5.0 mmol) in diphenyl ether (20 ml) was heated to 260° C. overnight. Then the temperature was raised to 270° C. After 69 h, the reaction mixture was cooled, and methanol (100 ml) was added. After stirring for 1 h, the product was filtered off, washed with methanol, and finally dried in vacuo overnight. Yield: Quantitative.

b) 1,2-bis(4-dodecylphenyl)-3,4,5,6-tetraphenylbenzene

1-dodecene (30.0 mmol) was slowly added to a 0.5 M solution of 9-borabicyclo[4.4.1] nonane in THF (65 ml), and the mixture was stirred at room temperature overnight. Then a solution of NaOH (45.0 mmol) in water (15 ml) was slowly added, and the mixture was stirred for 20 min. The dibromide (2.5 mmol) was added, followed by Pd(dppf)Cl2 (80 mg). The reaction mixture was stirred at room temperature for 5 h. Then TLC analysis indicated incomplete reaction, and the temperature was increased. After boiling at reflux overnight, the reaction mixture was cooled to room temperature and concentrated in vacuo. The residue was dissolved in CH2CL2, washed with water and with brine, and then dried (Na2SO4). The dry solution was diluted with hexane, filtered thru celite, and concentrated in vacuo. Finally the product was purified by flash chromatography (SiO2, pentane w/5-10% CH2Cl2). Yield: 80%.

c) 2,5-didodecylhexaperihexabenzocoronene

The didodecylhexaphenylbenzene (1.5 mmol) was dissolved in dry CH2Cl2 (750) ml, and argon was bubbled thru the solution for 15 min. Then anhydrous FeCl3 (45 mmol) dissolved in nitromethane (15 ml) was added, and the mixture was stirred at room temperature while being bubbled with argon. After 75 min, the reaction mixture was poured into methanol (1 l). The reaction mixture was then concentrated in vacuo to remove most of the CH2Cl2, and the precipitated product was filtered off, washed thoroughly with dilute hydrochloric acid and with methanol, and then dried in vacuo. The crude product was dissolved in hot THF, precipitated once again with methanol, and finally dried in vacuum overnight. Yield: 55%.

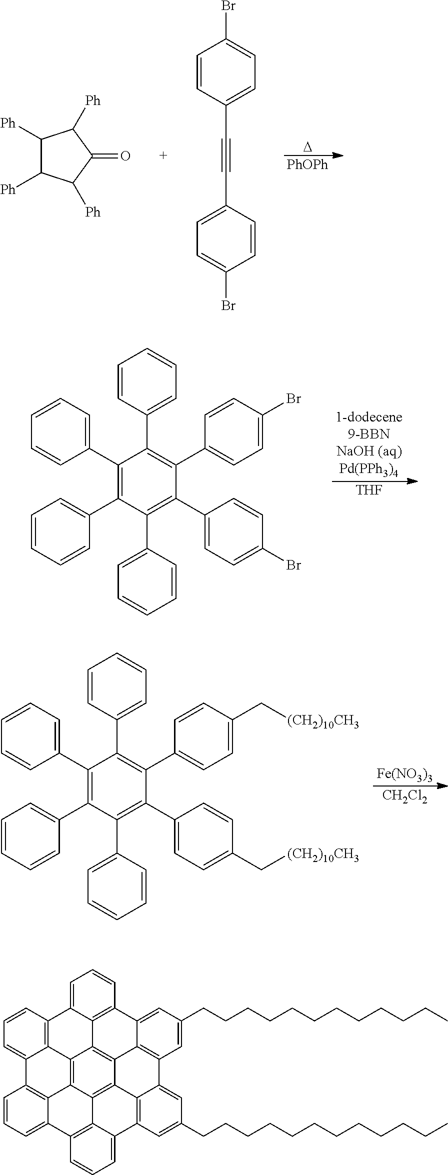

Proof of Structure

Confirmation of expected structure was first sought for by solid state NMR. ¹H-MAS was performed to qualitatively evaluate the relative contribution of aromatic and aliphatic protons to the total resonance signal. The following graph shows that aromatic and aliphatic contributions are well separated. Deconvolution and integration of peaks leads to:
Aliphatic protons: 72%,
Intermediate protons: 3%
Aromatic protons: 25%

The match with the expected structure (50-16 ratio between aliphatic and aromatic protons) appears to be fairly quantitative.

1 H-MAS solid state NMR of model asphaltenes (20 kHz).

Further characterization was performed with an Agilent 6500 series Accurate-Mass Quadrupole Time of Flight Mass Spectrometer (QTOF). Atmospheric pressure Photoionization (APPI) was used as an ion source. The signal primarily consists of a set of 5 peaks starting at 858.58 Da (expected molar weight of di-dodecylhexaperihexabenzocoronene) and separated by 1 Da. They correspond quantitatively to the isotopic distribution for a mixture of 43% radical and 57% protonated cations. A secondary set of similar peaks starting at 893.48 Da was attributed to the substitution of one hydrogen atom by one chlorine atom on the aromatic core. The chlorinated impurities are probably due to the use of FeCl₃ for the final cyclization/oxidation step. In any case, actually observed quantities correspond to traces (0.25% of hydrogen substituted by chlorine) and should not change the overall results. MS-MS fragmentation experiments were also conducted. When collision energy is increased, peaks appear at masses corresponding to the mono-dodecyl-hexaperihexabenzocoronene (691.33 Da) and hexaperihexabenzocoronene (523.14 Da). This reveals the progressive ablation of alkyl chains and confirms the structure of both the poly-aromatic core and the side alkyl chains.

Primary mass peaks of model asphaltenes (QTOF APPI-200V fragmentor voltage).

collision induced fragmentation of model asphaltenes (QTOF APPI-MS/MS CID).

### Example 2

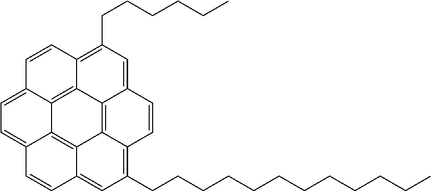 [SMILES: CCCCCCCCCCCCc1cc2ccc3ccc4ccc5c(CCCCCC)cc6ccc1c1c2c3c4c5c61]

Model asphaltene (˜500 g/mol) (see also FIG. 3)

Synthesis can be performed by alkylation of commercial coronene either directly (Friedel-Craft reaction) or with intermediate bromination.

### CITATION LIST

Energy & Fuels 2006, 20, 2439-2447
H. Groenzin, O. C. Mullins, Energy & Fuels 14(2000) 677-684
Andrews, A. B.; McClelland, A.; Korkeila, A.; Demidov, A.; Krummel, A.; Mullins, O. C.; Chen Z. Molecular Orientation of Asphaltenes and PAH Model Compounds in LangmuirBlodgett Films Using Sum Frequency Generation Spectroscopy. Langmuir, 2011, 27 (10), pp 6049-6058

1-10. (canceled)

11. A synthetic compound comprising a pericondensed aromatic core with 4-12 aromatic rings substituted with 1-2 linear alkyl chains containing 5-12 carbon atoms, having a molecular mass between 300-800 g/mol and wherein the proportion of aliphatic carbon atoms ranges from 30-60% of the total number of carbon atoms in the molecule.

12. A compound according to claim 11, wherein the range of molecular mass is between 600-800 g/mol, preferably about 750 g/mol.

13. A compound according to claim 11, wherein the linear alkyl chains contain 5-12 carbon atoms, preferably 7-8 carbon atoms.

14. A compound according to claim 11 which is:

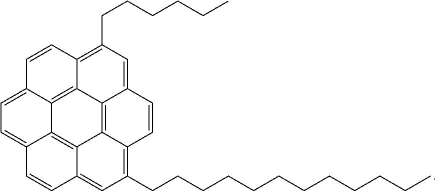 [SMILES: CCCCCCCCCCCCc1cc2ccc3ccc4ccc5c(CCCCCC)cc6ccc1c1c2c3c4c5c61]

15. Compounds according to claim 11 further substituted by carboxylic functions located on the terminal carbon of the alkyl chain.

16. Mixtures of the compounds according to claim 11 dissolved in aromatic or aliphatic base oils or mixtures thereof.

17. Mixtures according to claim 16 wherein the alkylated graphenes constitute 10 to 200 ppm (w/w).

18. Use of the mixtures according to claim 16 as multiphase flow test fluids.

19. Use according to claim 18 in separation tests.

20. Use according to claim 18 in the oil and gas industry.

21. A synthetic compound comprising a pericondensed aromatic core with 4-13 aromatic rings substituted with 1-2 linear alkyl chains containing 5-20 carbon atoms, having a molecular mass between 300-860 g/mol and wherein the proportion of aliphatic carbon atoms ranges from 30-60% of the total number of carbon atoms in the molecule.

22. A compounds according to claim 21 which is:

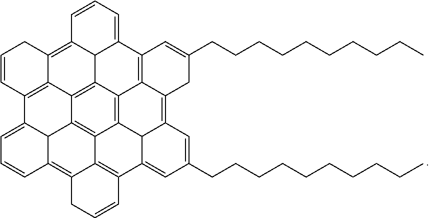 [SMILES: CCCCCCCCCCC1=CC2=C3CC(CCCCCCCCCC)=CC4=C3c3c5c6c7c8c3C2C(=C1)C1=C8C(=C2C=CC=C(C3=C6C(=C6C=CC=C4C65)CC=C3)C27)CC=C1]

23. Compounds according to claim 21 further substituted by carboxylic functions located on the terminal carbon of the alkyl chain.

24. Mixtures of the compounds according to claim 21 dissolved in aromatic or aliphatic base oils or mixtures thereof.

25. Mixtures according to claim 24 wherein the alkylated graphenes constitute 10 to 200 ppm (w/w).

26. Use of the mixtures according claim 24 as multiphase flow test fluids.

27. Use according to claim 26 in separation tests.

28. Use according to claim 26 in the oil and gas industry.
